(19) United States
(12) Patent Application Publication
(10) Pub. No.: US 2022/0183879 A1
(43) Pub. Date: Jun. 16, 2022

(54) DEVICE AND METHOD FOR MANAGING OBSTRUCTIVE SLEEP APNEA

(71) Applicants: Nazmi PEYMAN, Richmond, VA (US); EDMOND ZAHEDI, Burnaby BC (CA)

(72) Inventors: Nazmi PEYMAN, Richmond, VA (US); EDMOND ZAHEDI, Burnaby BC (CA)

(21) Appl. No.: 17/548,482

(22) Filed: Dec. 10, 2021

Related U.S. Application Data
(60) Provisional application No. 63/123,966, filed on Dec. 10, 2020.

(51) Int. Cl. A61F 5/56; A61F 2/08; A61F 2/48

(52) U.S. Cl. CPC A61F 5/566; A61F 2/08; A61F 2250/0002; A61F 2210/009; A61F 2/482

(57) ABSTRACT

A device and method for managing obstructive sleep apnea in patients. The device includes a first implantable member and a second implantable member, both configured to generate a magnetic field. The first implantable member is implanted in a base of a tongue and the second implantable member is implanted in a soft palate such that like magnetic poles of the first implantable member and the second implantable member face each other resulting in repulsion between the first implantable member and the second implantable member. The repulsive force between the first implantable member and the second implantable member can open the respiratory airway and keep it open.

### CROSS-REFERENCE TO RELATED APPLICATIONS

This application claims priority from the U.S. provisional patent application Ser. No. 63/123,966, filed on Dec. 10, 2020, which is incorporated herein by reference in its entirety.

### FIELD OF INVENTION

The present invention relates to a medical device, and more particularly, the present invention relates to an implantable device for managing obstructive sleep apnea.

### BACKGROUND

Obstructive Sleep Apnea (OSA) is a major cause of morbidity and mortality. It has been linked to a significant increase in the incidence of hypertension, cardiac disease, and cerebrovascular accidents. Besides creating discomfort for a partner because of snoring, patients usually suffer from daytime sleepiness and fatigue as their night sleep can be disrupted up to a few hundred times during an 8-hour sleep period.

Currently, the first line of treatment for OSA is using Continuous positive airway pressure therapy (CPAP) devices, where the patient has to wear a mask during sleep. Through this mask, the patient is subjected to a continuous positive airflow keeping the respiratory airway open. The CPAP mask itself is not well tolerated by the patients as it may itself disrupt sleep. This is because wearing the mask during sleep feels uncomfortable, leading to a low adherence rate. In severe cases, upper airway surgery is recommended, aiming at removing some of the tissues, which may or may not be successful in treating OSA and in itself may create some other complications. Another treatment is hypoglossal nerve stimulation (HNS), based on the principle of electrically stimulating the corresponding nerves. This approach involves surgically implanting an internal pulse generator device in the chest and connecting associated leads to the site of stimulation in the upper respiratory tissues. The eligibility criteria disallow usage for obese patients. Remarkably, obese subjects are the most affected by OSA.

A need is therefore appreciated for a novel mechanism to prevent obstructive sleep apnea in patients that is comfortable in use and thus results in positive patient compliance.

In this patent, an implantable system to prevent sleep apnea is described, which acts by creating a repulsive force between the involved respiratory airway tissues whenever apnea is detected. This repulsive force is generated by two opposing magnetic fields and is activated at the onset of sleep apnea.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a device and method for managing obstructive sleep apnea in patients.

It is another object of the present invention that the device can be implanted.

In one aspect, disclosed is a device and method to manage obstructive sleep apnea in patients suffering from it. The device can include a first implantable member and a second implantable member, wherein both the first implantable member and the second implantable member can generate a magnetic field, such that when like poles of the first implantable member and the second implantable member face each other, the first implantable member and the second implantable member repel each other due to the opposing magnetic field.

In one aspect, the first implantable member can be implanted in a base of the tongue and the second implantable member can be implanted in a soft palate, such as the first implantable member and the second implantable member repel each other due to the magnetic field, and the repulsion between the first implantable member and the second implantable member can keep the airway open.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 illustrates the disclosed device and method for managing obstructive sleep apnea, the device includes two permanent magnets, according to an exemplary embodiment of the present invention.

FIG. 2 illustrates the disclosed device and method for managing obstructive sleep apnea, wherein the device includes a solenoid that can be implanted in a tongue and a permanent magnet that can be implanted in a soft palate, according to an exemplary embodiment of the present invention.

FIG. 3 illustrates the disclosed device and method for managing obstructive sleep apnea, wherein the device includes a permanent magnet that can be implanted in the tongue and a solenoid that can be implanted in a soft palate, according to an exemplary embodiment of the present invention.

FIG. 4 illustrates the disclosed device and method for managing obstructive sleep apnea, wherein the device includes two solenoids, according to an exemplary embodiment of the present invention.

FIG. 5 illustrates the disclosed device and method for managing obstructive sleep apnea, wherein the device includes a biological solenoid that can be implanted in the tongue and a permanent magnet that can be implanted in a soft palate, according to an exemplary embodiment of the present invention.

FIG. 6 illustrates the disclosed device and method for managing obstructive sleep apnea, the device includes a permanent magnet that can be implanted in a tongue and a biological solenoid that can be implanted in a soft palate, according to an exemplary embodiment of the present invention.

FIG. 7 illustrates the disclosed device and method for managing obstructive sleep apnea, wherein the device can include two biological solenoids, according to an exemplary embodiment of the present invention.

FIG. 8 illustrates the disclosed device and method for managing obstructive sleep apnea, wherein the device includes a solenoid that can be implanted in a tongue and a biological solenoid that can be implanted in a soft palate, according to an exemplary embodiment of the present invention.

FIG. 9 illustrates the disclosed device and method for managing obstructive sleep apnea, wherein the device can include a biological solenoid that can be implanted in a tongue and a solenoid that can be implanted in a soft palate, according to an exemplary embodiment of the present invention.

FIG. 10 illustrates the device including a wireless power source, an apnea detector, a biological solenoid, and a solenoid, according to an exemplary embodiment of the present invention.

### DETAILED DESCRIPTION

The accompanying figures, which are incorporated herein, form part of the specification and illustrate embodiments of the present invention. Together with the description, the figures further explain the principles of the present invention and to enable a person skilled in the relevant arts to make and use the invention.

Furthermore, in the following description of embodiments, numerous specific details are set forth in order to provide a thorough understanding of the present technology. However, the present technology may come together in the form of a complete assembly without these specific details. In some instances, well-known methods, procedures, devices, and circuits have not been described in detail as not to unnecessarily obscure aspects of the present embodiments.

To simplify the descriptions, in terms of North and South magnetic poles, only one configuration has been described. It should be noted that the selection of the North/South poles for the magnetic fields is arbitrary and can be reversed. As such, in this document, all North poles can be replaced by South poles, and all South poles be replaced by North poles without affecting the overall result.

The location where the magnetic generating field elements are inserted is also one particular embodiment in this document, namely the soft palate and base of the tongue. It is understood that other tissues potentially involved in airway obstruction can also be selected to this end, but for the sake of simplifying this document are not repeated here.

Disclosed is a device and method for managing obstructive sleep apnea in patients suffering from it. The disclosed device and method can prevent the airway from collapsing during sleep, and without disturbing the sleep of the patient. The disclosed device can be implanted and thus does not interface or cause discomfort to the patient while sleeping. One or more aspects of the disclosed device can be automated and may not require any intervention from the patient for operating the device. Overall, patient compliance can be improved, and the incidents of medical emergencies can be significantly lowered in patients due to obstructive sleep apnea. The disclosed device can be adapted to people of different ages and body structures, such as for both thin and obese patients.

In one implementation, the disclosed device can include a first implantable member and a second implantable member wherein both the first implantable member and the second implantable member can be implanted in body tissues or organs related to the respiratory airway that are affected by obstructive sleep apnea or responsible for blocking the airway, such as a base of a tongue and a soft palate. Both the first implantable member and the second implantable member can generate magnetic fields, such that when the two members are positioned having their like magnetic poles facing each other, the two members repel each other. The repulsion of the two members when implanted in the base of the tongue and the soft palate can prevent the airway from collapsing i.e., can keep the airway open. It is understood that the magnetic field generated can be of sufficient strength to keep the airway open and may not be more that can cause any discomfort.

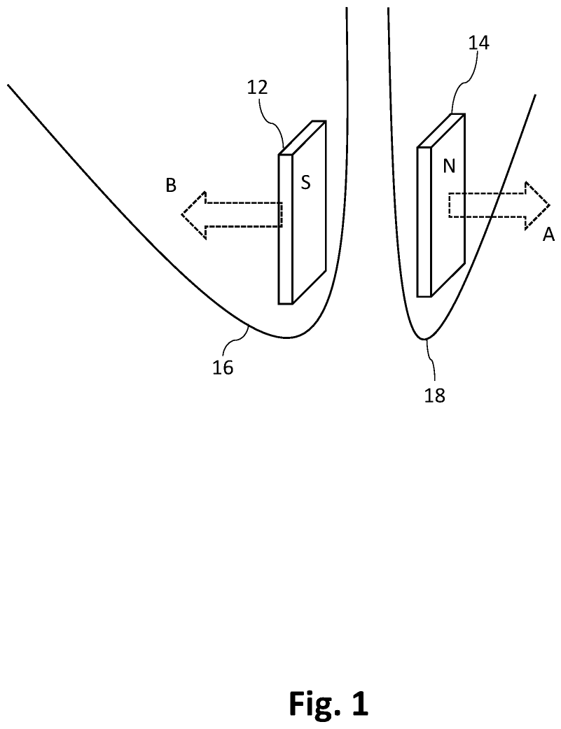

Referring to FIG. 1, which illustrates one implementation of the disclosed device and method. FIG. 1 shows the device including two permanent magnets, the first permanent magnet 12 as the first implantable member and a second permanent magnet 14 as the second implantable member. The first permanent magnet 12 can be seen implanted in a base of a tongue 16 and the second permanent magnet 14 can be seen implanted in a soft palate 18. Like poles of the two magnetic members can face each other. For example, FIG. 1 shows the south poles of the two magnetic members facing each other. It is understood that the polarity can be reversed by having the north poles facing each other without departing from the scope of the present invention. The opposing magnetic fields are shown by arrows A and B which are shown pointed in opposite directions illustrating the repulsive magnetic fields.

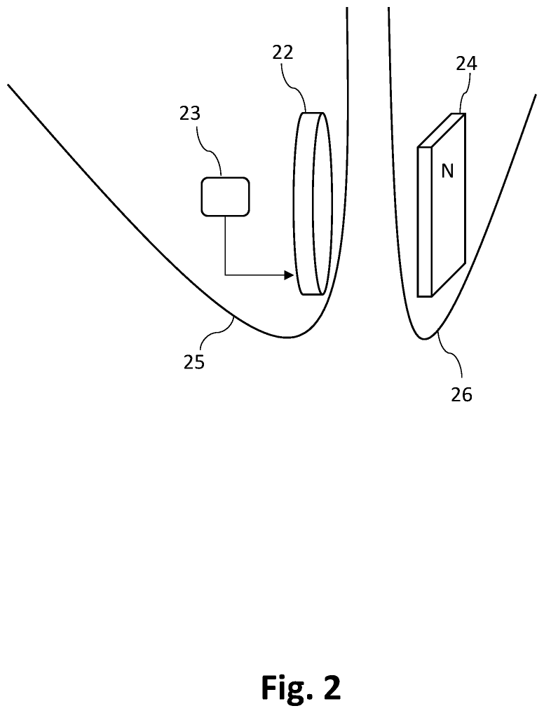

Referring to FIG. 2 shows another implementation of the disclosed device and method. The device can include a solenoid 22 that can be powered by a power source 23 to generate the magnetic field. The second implantable member can be a permanent magnet 24. The solenoid 22 is shown to be implanted in the base of the tongue 25 and the permanent magnet 24 is shown implanted in the soft palate 26. Once powered by a source 23 of electrical energy, the solenoid 22 can generate a magnetic field in such a way that its South pole (S) faces the South pole (S) of the permanent magnet, or its North pole (N) faces the North pole (N) of the permanent magnet. The magnetic fields generated by the two members oppose each other, hence creating a repulsive force that keeps the respiratory airway open. The solenoid can be an electrical solenoid made from electrical wires.

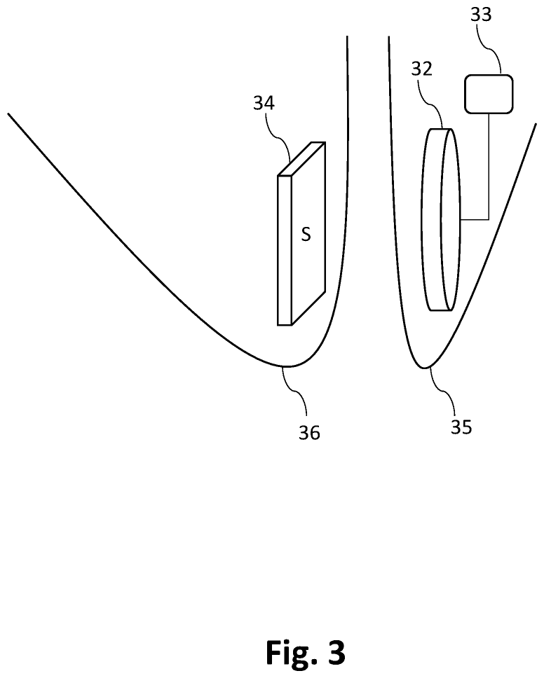

Referring to FIG. 3 shows another implementation of the device and method. FIG. 3 shows a solenoid 32 and a permanent magnet 34 used to generate the magnetic fields. Once powered by a source 33 of electrical energy, the solenoid generates a magnetic field in such a way that its South pole (S) faces the South pole (S) of the permanent magnet. The magnetic fields generated by two members oppose each other, hence creating a repulsive force that keeps the respiratory airway open. The permanent magnet 34 can be implanted in the base of the tongue 36, whereas the solenoid 32 can be implanted in the soft palate 35.

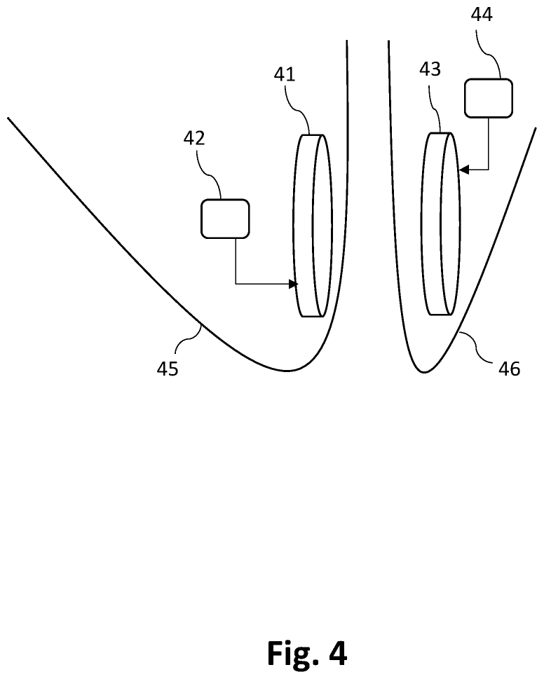

Referring to FIG. 4 shows another implementation of the device and method. FIG. 4 shows two solenoids, i.e., a first solenoid 41 as the first implantable member and a second solenoid 43 as the second implantable member. The first solenoid can be connected to a power source 42 and the second solenoid can be connected to a power source 44. Once powered, the two solenoids can generate magnetic fields in such a way that their South poles (S) face each other. These magnetic fields oppose each other, hence creating a repulsive force that keeps the respiratory airway open. The first solenoid 41 is shown implanted in the base of the tongue 45, and the second solenoid 43 is shown implanted in the soft palate 46.

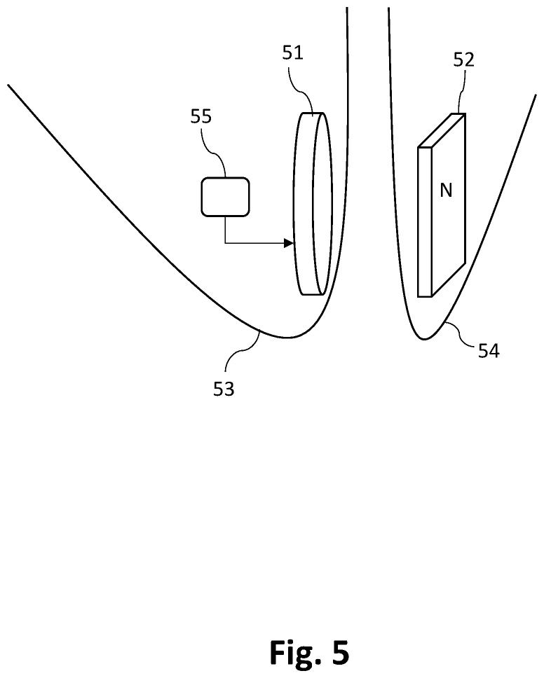

Referring to FIG. 5 shows another implementation of the device and method. FIG. 5 shows a biological solenoid 51 and a permanent magnet 52 that can be used to generate the magnetic fields. The biological solenoid can be grown out for example from stem cells allowing an annular-shaped formation for an electrically conductive tissue such as an axon to be formed. Once powered by a source 55 of electrical energy, the biological solenoid generates a magnetic field in such a way that its South pole (S) faces the South pole (S) of the permanent magnet. The magnetic fields generated by the two members can oppose each other, hence creating a repulsive force that keeps the respiratory airway open. FIG. 5 shows the biological solenoid 51 implanted in the base of the tongue 53 and the permanent magnet 52 implanted in the soft palate 54.

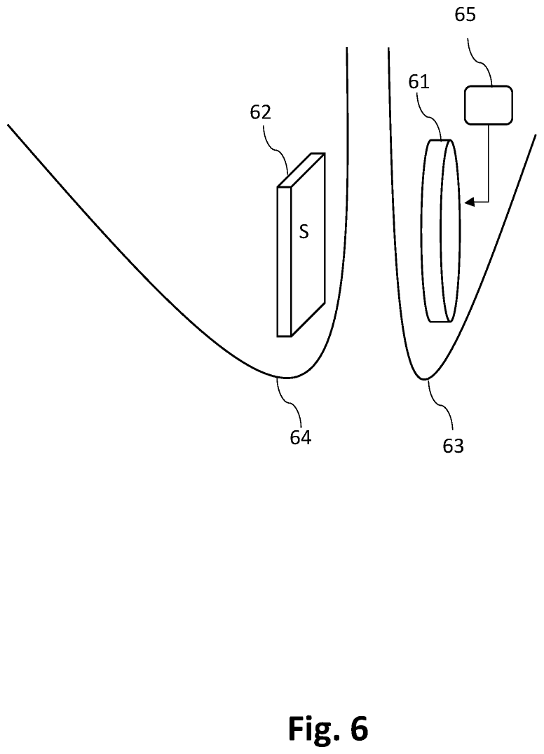

Referring to FIG. 6 shows another implementation of the device and method. FIG. 6 shows a biological solenoid 61 and a permanent magnet 62 are used to generate the magnetic fields. The biological solenoid can be powered by a source 65 of electrical energy to generate a magnetic field in such a way that its South pole (S) faces the South pole (S) of the permanent magnet. The magnetic fields generated by each of these two elements oppose each other, hence creating a repulsive force that keeps the respiratory airway open. FIG. 6 shows the biological solenoid implanted in the soft palate 63 and the permanent magnet 62 implanted in the base of the tongue 64.

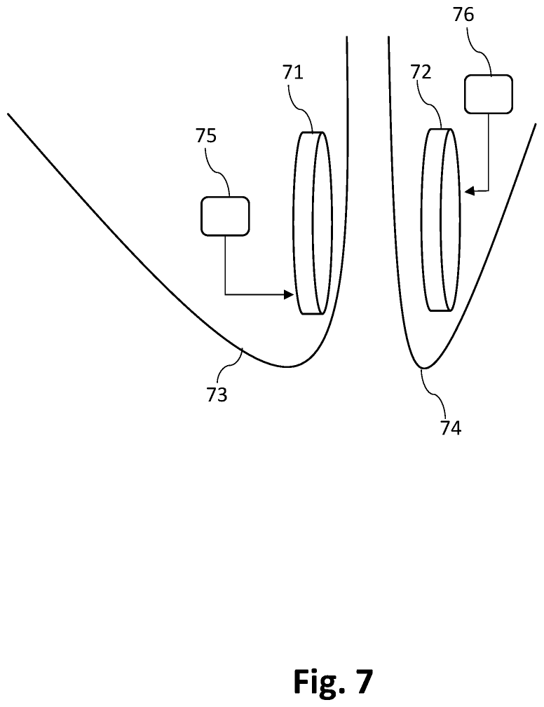

Referring to FIG. 7 shows another implementation of the device and method. FIG. 7 shows a first biological solenoid 71 and a second biological solenoid 72 that can be used to generate the magnetic fields. The first biological solenoid 71 is shown powered by a first power source 75 and the second biological solenoid powered by a second power source 76. Once powered, the two biological solenoids can generate a magnetic field in such a way that their South poles (S) face each other. These magnetic fields oppose each other, hence creating a repulsive force that keeps the respiratory airway open. The first solenoid 71 is shown implanted in the base of the tongue 73 and the second biological solenoid 72 implanted in the soft palate 74.

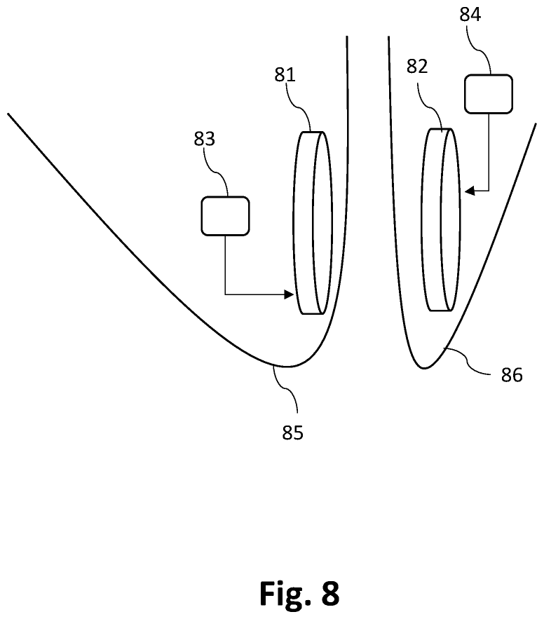

Referring to FIG. 8 shows another implementation of the device and method. FIG. 8 shows a biological solenoid 82 and a non-biological solenoid 81 that can be used to generate magnetic fields. The biological solenoid 82 can be grown out for example stem cells allowing an annular-shaped formation for an electrically conductive tissue such as an axon to be formed. The biological solenoid 82 can be powered by a first power source 84 and the non-biological solenoid 81 can be powered by a second power source 83. Once powered by a source of electrical energy, the biological and non-biological solenoids generate a magnetic field in such a way that their South poles (S) face each other. The magnetic fields generated by each of these two elements oppose each other, hence creating a repulsive force that keeps the respiratory airway open. FIG. 8 shows the biological solenoid 82 implanted in the soft palate 86 and the non-biological solenoid 81 implanted in the base of the tongue 85.

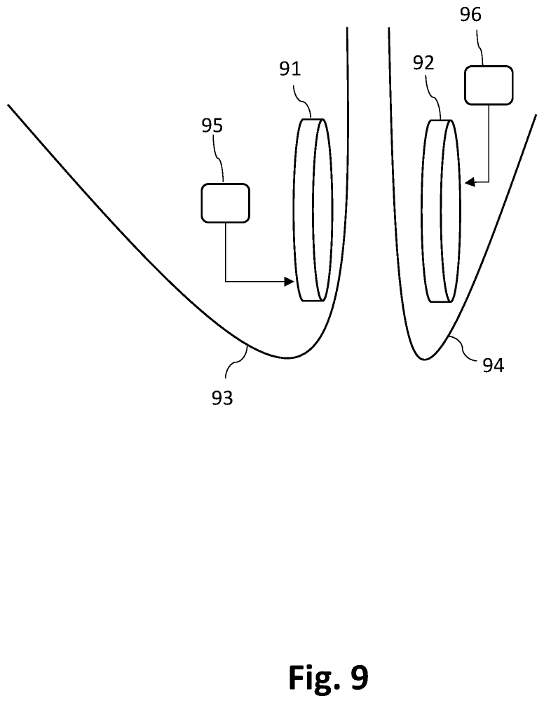

Referring to FIG. 9 shows another implementation of the device and method. FIG. 9 shows a biological solenoid 91 and a non-biological solenoid 92 that can be used to generate the magnetic fields. The biological solenoid 91 can be grown out for example stem cells allowing an annular-shaped formation for an electrically conductive tissue such as an axon to be formed. The biological solenoid 91 can be powered by a first power source 95 and the non-biological solenoid 92 can be powered by a second power source 96. Once powered by a source of electrical energy, the biological and non-biological solenoids can generate a magnetic field in such a way that their South poles (S) face each other. The magnetic fields generated by each of these two elements oppose each other, hence creating a repulsive force that keeps the respiratory airway open. FIG. 9 shows the biological solenoid 91 implanted in the base of the tongue 93 and the non-biological solenoid 92 implanted in the soft palate 94.

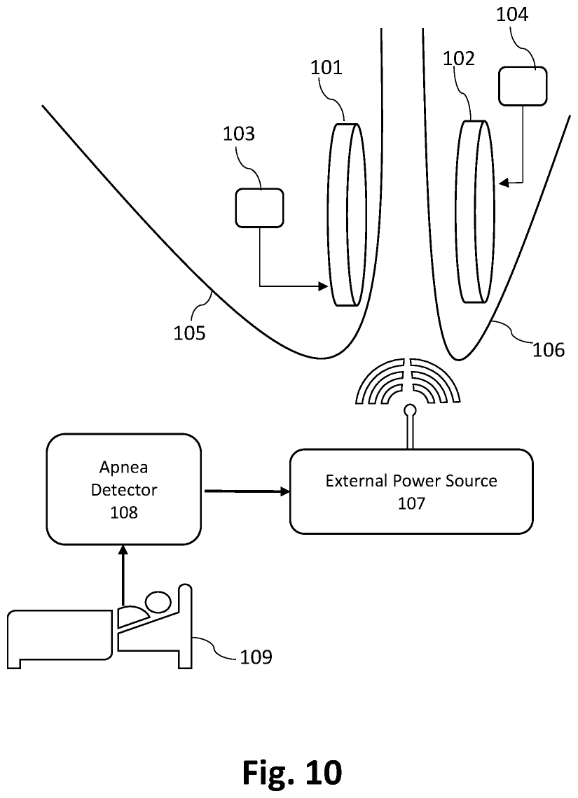

Referring to FIG. 10 which shows another implementation of the device and method which additionally shows control and power circuitry. FIG. 10 shows a biological solenoid 101 and non-biological solenoid 102 are used to generate the magnetic fields. Two power sources 103 and 104 can power the biological solenoid 101 and the non-biological solenoid 102 respectively. The device can further include an apnea detector for detecting an onset of apnea. The apnea detector 108 is shown connected to a patient on bed 109 to detect respiration of the patient and as soon as apnea can be detected, the two power sources can be activated. FIG. 10 also shows an external power source 107 that can supply power to the two power sources 103 and 104. This power source transmits wireless power to the implanted coils, resulting in the generation of the magnetic fields as described in the previous paragraphs.

In one implementation, the disclosed device and method can provide for automated operation using one or more sensors that can detect the onset of apnea in the patient, and in response to a signal from the sensor indicating the onset of apnea, the first implantable member and the second implantable member can be activated to generate repulsive magnetic fields which opens the airway and keeps it open. In one implementation, the two magnetic members can remain activated for a pre-determined duration and then can be deactivated, and upon deactivation of the two members, the sensors can be activated again. This process can be repeated for autonomous management of obstructive sleep apnea in patients. The sensors can be any sensors that can detect the onset of apnea. The sensors can be implantable or can be external to the body. Suitable examples of sensors can include proximity sensors, accelerometers, gyroscopes, and like that can detect a change in spatial positions or closeness between two objects. Vibration sensors can also be used that can detect vibrations produced in body tissues due to snoring or blockage of the airway. Noise sensors can also be used that can detect a change in breathing voices or snoring to detect the onset of apnea. Similar, respiration sensors can also be used to detect the onset of apnea. It is understood that all such sensors and other sensors known to a skilled person for detecting the onset of apnea are within the scope of the present invention. Suitable logical circuitry can also be provided and can be implanted or can be external to the human body. The logical circuitry can connect to the sensors and the two magnetic/implantable members through a wired or wireless connection. The logical circuitry upon receiving a signal from the sensors indicating the onset of apnea can activate the two magnetic members to generate the magnetic field, thus keeping the airway open. The two magnetic members (implantable members) can be activated and deactivated in a variety of ways, such as turning the power supply on and off. The predetermined duration for keeping the two magnetic members active can also be set in the logical circuitry. It is understood that the logic circuitry can be optional, and the sensors can be directly coupled to the two magnetic members for activation of the two magnetic members.

The different components of the device can be powered by single or multiple power sources. One or more of such power sources may be implanted. One or more power sources can be wireless. Other suitable examples of power sources can include implantable batteries. Any such means of powering the sensors, the logical circuitry, the first implantable member, and the second implantable member are within the scope of the present invention.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A device for managing obstructive sleep apnea, the device comprises:
a first implantable member configured to generate a magnetic field; and
a second implantable member configured to generate a magnetic field, wherein the first implantable member and the second implantable member are configured to be implanted in body tissues related to airway obstruction, wherein the first implantable member and the second implantable member are configured such that when implanted in the body tissues, the first implantable member and the second implantable member repel each other due to the magnetic field.

2. The device according to claim 1, wherein the device further comprises one or more sensors configured to detect an onset of apnea.

3. The device according to claim 1, wherein the first implantable member and the second implantable member are permanent magnets.

4. The device according to claim 1, wherein the first implantable member and the second implantable member are solenoids.

5. The device according to claim 1, wherein the first implantable member and the second implantable member are biological solenoids.

6. The device according to claim 1, wherein the first implantable member is a solenoid, and the second implantable member is a permanent magnet.

7. A method for managing obstructive sleep apnea, the method comprising the steps of:
providing a first implantable member and a second implantable member both configured to generate a magnetic field; and
implanting the first implantable member in a first body tissue and the second implantable member in a second body tissue, such that like poles of the first implantable member and the second implantable member face each other resulting in repulsion between the first implantable member and the second implantable member due to the magnetic field, wherein the repulsion between the first implantable member and the second implantable member keeps a respiratory airway open.

8. The method according to claim 7, wherein the first body tissue is a base of a tongue.

9. The method according to claim 8, wherein the second body tissue is a soft palate.

10. The method according to claim 9, wherein the first implantable member is a permanent magnet, and the second implantable member is a permanent magnet.

11. The method according to claim 7, wherein the method further comprises the steps of:
providing one or more sensors configured to detect an onset of apnea;
receiving a signal from the one or more sensors indicating the onset of apnea; and
upon receiving the signal, activating the first implantable member and the second implantable member to generate the magnetic field.

12. The method according to claim 11, wherein the first implantable member is an electrical solenoid, and the second implantable member is an electrical solenoid.

13. The method according to claim 11, wherein the first implantable member is a permanent magnet, and the second implantable member is an electrical solenoid.

14. The method according to claim 11, wherein the first implantable member is a biological solenoid, and the second implantable member is an electrical solenoid.

15. The method according to claim 11, wherein the first implantable member is a biological electrical solenoid, and the second implantable member is a biological solenoid.

16. The method according to claim 11, wherein the step of activating further comprises activating an external wireless power supply to power the first implantable member and the second implantable member.

17. The method according to claim 11, wherein the first body tissue is a base of a tongue, and the second body tissue is a soft palate.

18. The method according to claim 7, wherein the first implantable member and the second implantable member are selected from a group consisting of a permanent magnet, an electrical solenoid, and a biological solenoid, wherein both the first implantable member and the second implantable member are not permanent magnets.
